(19) United States
(12) Patent Application Publication
(10) Pub. No.: US 2007/0254856 A1
(43) Pub. Date: Nov. 1, 2007

(54) Use of acetylsalicylic acid (ASA) in the application of a membrane lung

(76) Inventors: Alois Philipp, Pentling (DE); Franz-Xaver Schmid, Kelheim (DE); Dietrich Birnbaum, Regensburg (DE)

Correspondence Address: MORRISON & FOERSTER LLP, 12531 HIGH BLUFF DRIVE, SUITE 100, SAN DIEGO, CA, 92130-2040, US

(21) Appl. No.: 11/788,589

(22) Filed: Apr. 19, 2007

(30) Foreign Application Priority Data
Apr. 21, 2006 (DE) 10 2006 020 492.1

(51) Int. Cl. A61K 31/616

(52) U.S. Cl. 514/165

(57) ABSTRACT

Acetylsalicylic acid (ASA) is used as a medicament which is administered to a human patient in connection with a treatment of the patient with a membrane lung, in order to avoid or reduce a deposition of blood constituents in the membrane lung. The administration of the medicament involves ASA initially being administered once, several times or continuously each day in a therapeutically effective daily amount that lies below the risk range for analgesic efficacy of ASA.

### CROSS-REFERENCE TO RELATED APPLICATIONS

This application claims priority under 35 U.S.C. § 119(d) from German Patent Application No. 10 2006 020 492.1, filed Apr. 21, 2006. The content of the above patent application is incorporated by reference herein in its entirety.

### BACKGROUND OF THE INVENTION

1. Field of the Invention

The present invention relates to the production of a medicament which is administered to a human patient in connection with a treatment of the patient with a membrane lung, in order to avoid or reduce a deposition of blood constituents in the membrane lung.

An extracorporeal lung assist system based on a membrane lung is marketed by the Applicant under the name NovaLung iLA (interventional Lung Assist).

On the blood side, the NovaLung iLA is connected directly to the blood circulation of a patient by percutaneous arterial and venous cannulation. The NovaLung iLA makes it possible, without using a blood pump, to remove carbon dioxide from the blood being pulsatilely pumped from the patient's heart through the membrane lung and to oxygenate it under the limitations of the inflow of arterial blood.

The importance of the NovaLung iLA, and of membrane lungs and artificial lung systems generally, is to be seen against the fact that lung diseases are the third most common cause of death according to statistics from the World Health Organization. In cases of lung failure, the only possibility available at present is that of mechanical ventilation. This, however, does not constitute a lung assistance, because the diseased lung is not treated. Instead, it simply ensures the gas exchange necessary to life.

Although mechanical ventilation is able to maintain gas exchange in almost all patients, the unnatural positive airway pressure causes injury to the lungs and other organs, known as ventilator-associated lung injury (VALI). With the NovaLung iLA, it is possible for the first time, by means of ventilation outside the lung, to achieve a highly protective ventilation or, for example, a bridge to lung transplantation, and to avoid VALI.

Lung transplantation, however, represents an extremely complex medical procedure, and one that is also associated with a high risk to the patient. Besides the fact that this therapy concept is reserved exclusively to patients who have an isolated lung disease and are otherwise healthy, the long-term results are unsatisfactory. A further consideration is that, because of the small number of donor organs that become available each year across the world, only a small number of lung transplantation procedures can be performed, and this does not meet the actual demand.

Against this background, there is a significant need for a lung replacement procedure as a treatment that prolongs life (“destination therapy”). An extracorporeal or implantable lung assist system that can oxygenate the blood and can also remove carbon dioxide from the blood is therefore of very great value to patients. Such lung assist systems, however, can be used not only on patients who are unsuitable for a transplantation, but also on patients who are waiting for a lung transplantation and who, during the waiting period, develop critical lung failure that necessitates ventilation.

The fact that artificial lung assist systems such as the NovaLung iLA can be used in principle for this purpose has been shown in a clinical study—see Fischer et al.: BRIDGE TO LUNG TRANSPLANTATION WITH THE NOVEL PUMPLESS INTERVENTIONAL LUNG ASSIST DEVICE NOVALUNG, in J Thorac Cardiovasc Surg. (2006) 131 (3):719-723.

In the context of the above study, patients who developed lung failure that failed to respond to mechanical ventilation were successfully bridged to transplantation by means of the NovaLung iLA lung assist system.

At present, such systems can be used in intensive care units for a period of a few weeks. Longer use of the NovaLung iLA is not possible because of neointima formation and other factors, unless the membrane lung is regularly replaced.

Adverse side effects are known also from other clinical applications of organ support systems in which foreign surfaces come into contact with blood. Examples of such organ support systems are heart-lung machines with an oxygenator, mechanical blood pumps, haemodialysis and heart support systems, for example artificial hearts.

The contact between the blood and the non-physiological foreign surfaces in these cases leads to activation of the coagulation, complement and fibrinolysis system, and of other cellular constituents of the blood, which leads to thrombus formation on the foreign surfaces and, consequently, to a blocking of the gas exchange membrane, for example, used in the oxygenator. These effects lead, in just a short time, to a noticeable reduction in gas exchange performance and to a noticeable increase in the flow resistance of the membrane lung to the blood stream driven from the patient's heart through the membrane lung, with the result that the membrane lung has to be replaced after just a short period of use.

2. Related Prior Art

For heart-lung machines, it is known to counteract thrombus formation by means of anticoagulation, which is induced by administration of heparin. Alternative anticoagulants that can be used are hirudin, danaparoid sodium, ancrod or argatroban, all of which, however, can have serious side effects. See the inaugural dissertation by Schenck zu Schweinsberg: Management of anticoagulation during cardiopulmonary bypass in patients with heparin-induced thrombocytopenia, Gieβen 2004.

Glauber el al.: Reduction of haemorrhagic complications during mechanically assisted circulation with the use of a multi-system anticoagulation protocol, in Int. J. of Artificial Organs (1995) 18:649-655, describe a multi-medicament therapy for attenuating the continuous attack of foreign surfaces on the coagulation system. They propose the administration of dipyridamol together with heparin, aprotinin and aspirin.

To avoid thromboembolisms in mechanical heart support systems, Etz et al.: Analysis of platelet function during left ventricular support with the Incor and Excor system, in Heart Surg. Forum (2004) 7(5):E423-427, propose using an anticoagulation protocol with heparin, aspirin and clopidogrel.

Bhatt el al.: Clopidogrel and Aspirin versus Aspirin alone for the Prevention of Atherothrombic Events, in N. Engl. J. Med. (2006) describe how aspirin in daily doses of 75 to 162 mg reduces the risk of heart attack and stroke.

Dalen: Aspirin to prevent heart attack and stroke: what's the right dose?, in Am. J. Med. (2006) 119(3):198-202, describes how a daily dose of 160 mg aspirin is suitable for reducing the risk of heart attack and stroke, but also mentions that at these doses, and at a dose of 80 mg of aspirin daily, there is a danger of “severe bleeding”, that is to say bleeding with haemodynamic impairment and a need for transfusion.

EP 0 665 023 B1 describes a medical material that contains an antiplatelet agent, for example aspirin. Foreign surfaces of organ replacement systems are intended to be produced from this material in order to counteract the formation of blood clots. Upon contact with blood, the aspirin is intended to be released continuously and progressively, such that it has a concentration of 4 μg per ml of blood in the blood stream.

Against this background, an object of the present invention is to make available a medicament of the type mentioned at the outset which, while as far as possible avoiding the occurrence of side effects, permits the application of membrane lungs not only for short-term, but also for medium-term and long-term lung support and permits the application of an implantable lung assist system based on a membrane lung.

### SUMMARY OF THE INVENTION

According to the invention, this object is achieved by the fact that acetylsalicylic acid (ASA) is used in the aforementioned production, and that the administration of the medicament involves ASA initially being administered once, several times or continuously each day in a therapeutically effective daily amount that lies below the risk range for analgesic efficacy of ASA.

The object of the invention is achieved in full by this means.

The inventors of the present application have in fact found that administration of even small daily amounts of ASA substantially reduces or completely avoids the deposition of blood constituents on the gas exchange membranes of membrane lungs. The fact that this can be achieved just from administration of small daily doses of ASA was surprising and could not have been expected from the publications discussed above.

Therefore, aspirin is used as the sole therapeutic active substance in the production of the medicament.

The fact that heparin administration can be dispensed with in this context has the further advantage of avoiding heparin-induced thrombocytopenia (HIT), from which many patients in intensive care units suffer.

The invention is therefore based on the use of ASA administered to a human patient in connection with a treatment of the patient with a membrane lung in order to avoid or reduce a deposition of blood constituents in the membrane lung, wherein ASA is initially administered once daily, several times or continuously each day in a therapeutically effective amount that lies below the risk range for analgesic efficacy of ASA.

According to the invention, the daily dose of ASA lies below 500 mg, preferably below 100 mg, more preferably in the range of ca. 40 to ca. 60 mg, and it is preferably ca. 50 mg.

ASA is either administered orally at these doses, or a corresponding equivalent dose is applied intravenously.

The daily amount of ASA to be administered is therefore so low that known side effects of ASA, in particular severe bleeding, that is to say bleeding with haemodynamic impairment and a need for transfusion, are negligible.

Thus, the medication according to the invention permits, for the first time, a long-term use of extracorporeal or implantable lung assist systems, with the result that it is no longer necessary for a membrane lung used on a patient to be replaced, as has hitherto been the case, at short intervals.

Extracorporeal and, above all, implantable lung assist systems are therefore not only financially more attractive, they are in fact medically and ethically justified, especially as regards implantation.

Further advantages are set out in the description.

It will be appreciated that the aforementioned features and those still to be explained below can be used not only in the respectively cited combinations but also in other combinations or singly, without departing from the scope of the present invention.

### BRIEF DESCRIPTION OF THE DRAWINGS

An illustrative embodiment of the invention is explained in more detail in the following description with reference to the attached drawing, in which:

### DETAILED DESCRIPTION OF PREFERRED EMBODIMENTS

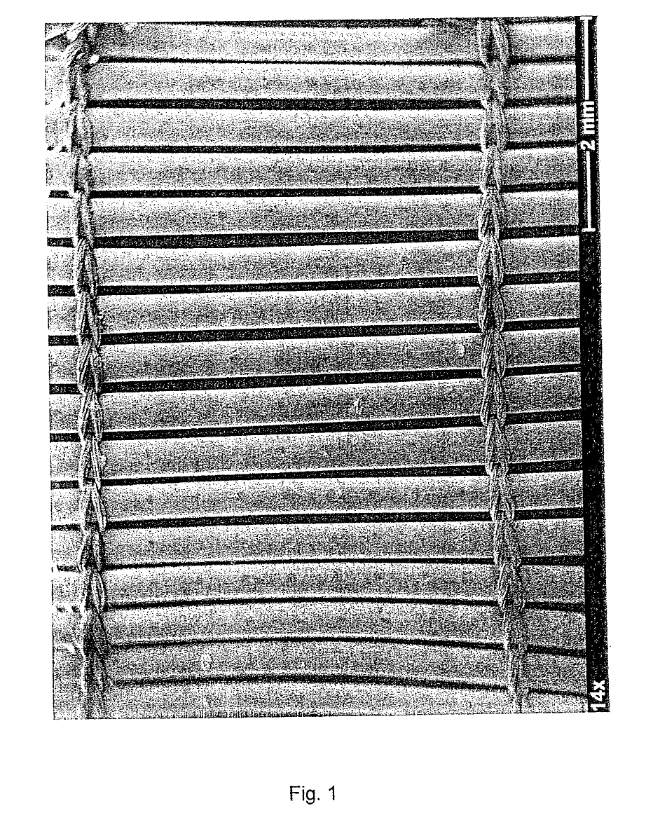

FIG. 1 shows a scanning electron microscope image of a gas exchange membrane from a membrane lung after a period of use of 14 days on a patient receiving oral administration of 50 mg ASA daily;

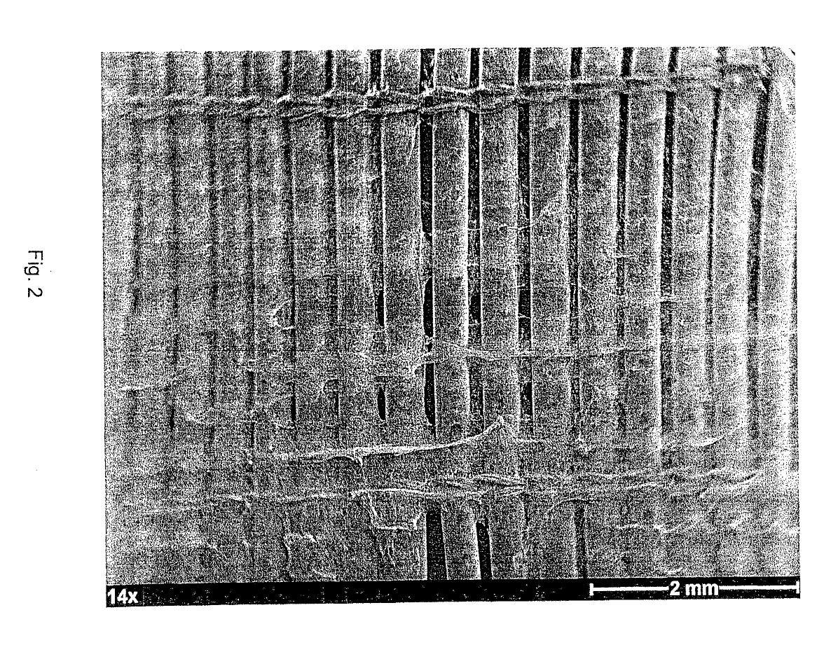

FIG. 2 shows an image as in FIG. 1 but without administration of ASA; and

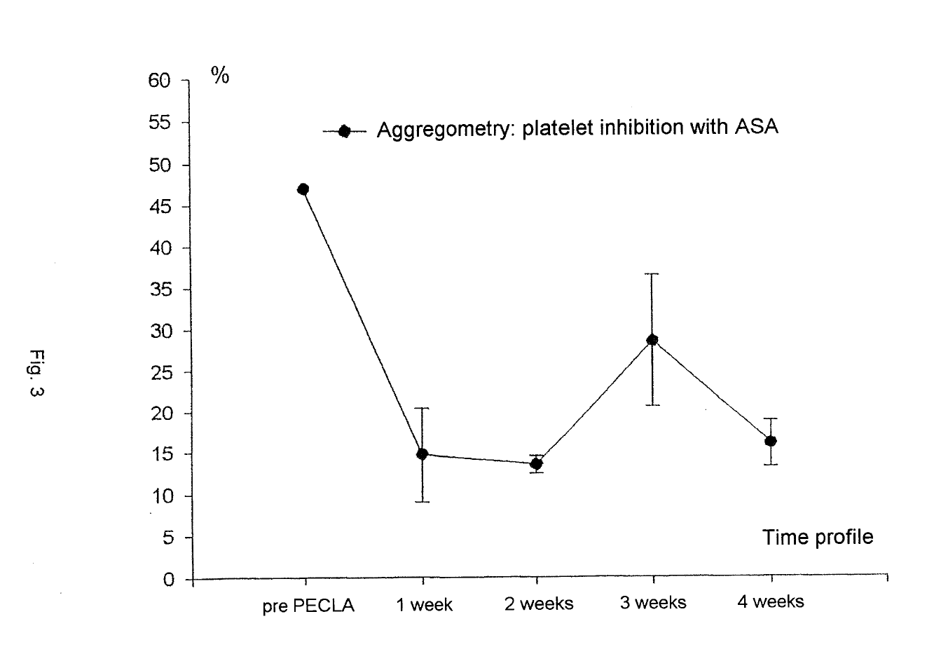

FIG. 3 shows the inhibition of platelet aggregation in a patient who received 50 mg of ASA daily in connection with the treatment with the NovaLung iLA.

A patient who required treatment with the NovaLung iLA was administered 50 mg of ASA daily for 1 week before application of the membrane lung. This administration of 50 mg of ASA daily was initially maintained during application of the membrane lung, but the medicament was discontinued one week before removal of the membrane lung. The membrane lung NovaLung iLA itself was used in the manner described by Fischer el al., loc cit.

It was possible in this way to leave the membrane lung on the patient for 2 weeks, without an appreciable reduction in gas exchange performance or an appreciable increase in the flow resistance of the membrane lung to the blood stream being driven from the patient's heart through the membrane lung.

FIG. 1 is a scanning electron microscope image of the gas exchange membrane from the membrane lung which was removed and dismantled after a period of use of 14 days on the patient thus treated and medicated. It will be seen that the gas exchange membrane shows no deposition of blood constituents, and that it could therefore have been used for much longer.

FIG. 2 shows an image comparable to FIG. 1. The patient was also treated for 14 days with the membrane lung, but did not receive medication with ASA. It will be seen that the gas exchange membrane has considerable deposits of blood constituents, for which reason it could not have been used for any longer without appreciable loss of performance and without endangering the patient.

The inventors of the present application are of the view that the effect of ASA administration on the useful life of the membrane lung on the patient can also be attributed to the fact that platelet aggregation is reduced by ASA, as is shown in FIG. 3 for the patients treated as above.

The platelet aggregation was determined using standard clinical methods.

The curve in FIG. 3 shows that the platelet aggregation had dropped to far below 20% within the first week of ASA administration, when the membrane lung was connected after 1 week. After a further week, ASA was discontinued, but the membrane lung was not removed until after an additional week.

During the second week of ASA administration, the platelet aggregation remained at its reduced value, and, after discontinuation of the medicament, it rose again only gradually. Two weeks after the start of treatment with the membrane lung, that is to say 1 week after discontinuation of the medicament, the platelet aggregation was still below 30%.

These results confirm overall that administration of ASA in a daily amount far below the analgesically effective amount of 500 mg, and even below the 100 mg recommended for prevention of heart attacks and strokes, permits long-term application of membrane lungs. Because of the small daily amount of ASA, the known side effects of ASA, in particular severe bleeding, that is to say bleeding with haemodynamic impairment and a need for transfusion, are negligible.

If ASA is administered once daily, several times or continuously each day, in a daily amount of 40 to 60 mg, preferably 50 mg, a membrane lung can be used on a patient for long periods of time without having to be replaced.

Therefore, what is claimed, is:

1. Use of acetylsalicylic acid (ASA) as medicament which is administered to a human patient in connection with a treatment of the patient with a membrane lung, in order to avoid or reduce a deposition of blood constituents in the membrane lung, wherein the administration of the medicament involves ASA initially being administered once, several times or continuously each day in a therapeutically effective daily amount that lies below the risk range for analgesic efficacy of ASA.

2. Use according to claim 1, wherein the daily administered amount of ASA lies below 500 mg, preferably below 100 mg.

3. Use according to claim 1, wherein the daily administered amount of ASA is in the range of ca. 40 to ca. 60 mg, preferably ca. 50 mg.

4. Use according to claim 1, wherein the administration of the medicament begins at least 1 day, preferably 5 to 7 days before application of the membrane lung.

5. Use according to claim 1, wherein the administration of the medicament is terminated at least 1 day, preferably 5 to 7 days before the end of the application of the membrane lung.

6. Use according to claim 1, wherein the administration of the medicament takes place in connection with a treatment with an extracorporeal or implantable lung assist system.

7. Use according to claim 1, wherein aspirin is used as the sole therapeutic active substance in the production of the medicament.

8. Use according to claim 1, wherein the medicament is administered orally, or is administered intravenously at a dose equivalent to the orally administered amount.

9. Use of ASA administered to a human patient in connection with a treatment of the patient with a membrane lung in order to avoid or reduce a deposition of blood constituents in the membrane lung, wherein ASA is initially administered once daily, several times or continuously each day in a therapeutically effective daily amount that lies below the risk range for analgesic efficacy of ASA.
